(19) United States
(12) Patent Application Publication
(10) Pub. No.: US 2005/0234543 A1
(43) Pub. Date: Oct. 20, 2005

(54) Plug for use in left atrial appendage

(75) Inventors: Erik Glaser, Waltham, MA (US); Todd A. Peavey, Cambridge, MA (US)

Correspondence Address: WILMER CUTLER PICKERING HALE AND DORR LLP, 60 STATE STREET, BOSTON, MA, 02109, US

(73) Assignee: NMT Medical, Inc., Boston, MA (US)

(21) Appl. No.: 11/093,170

(22) Filed: Mar. 29, 2005

Related U.S. Application Data
(60) Provisional application No. 60/557,611, filed on Mar. 30, 2004.
(60) Provisional application No. 60/557,484, filed on Mar. 30, 2004.

(51) Int. Cl. A61M 29/00

(52) U.S. Cl. 623/1.42; 606/191; 604/104

(57) ABSTRACT

A plug or insert occludes the left atrial appendage (LAA), thus preventing blood from entering. The plug is formed in one piece without separately movable parts, and may be monolithic. A drug coating can be provided, with or without a plug.

### CROSS-REFERENCE TO RELATED APPLICATIONS

This application claims priority to provisional application Ser. Nos. 60/557,611, filed Mar. 30, 2004; and 60/557,484, filed Mar. 30, 2004; each of which is incorporated herein by reference.

### BACKGROUND OF THE INVENTION

Arrhythmias are abnormal heart rhythms that may cause the heart to function less effectively. Atrial fibrillation (AF) is the most common abnormal heart rhythm. In AF, the two upper chambers of the heart (i.e., the atria) quiver rather than beat and, consequently, fail to entirely empty of blood. As blood stagnates on the walls of the atria, it may form thrombi (i.e., clots). Under certain circumstances, these thrombi can re-enter the circulation and travel to the brain, causing a stroke or a transient ischemic attack (TIA).

Research has indicated that as many as ninety (90) percent of all thrombi formed during AF originate in the left atrial appendage (LAA). Referring to FIG. 15, the LAA 111 is a remnant of an original embryonic left atrium that develops during the third week of gestation. It is located high on the free wall of the left atrium 112. Long, tubular, and hook-like in structure, the LAA 111 is connected to the left atrium 112 by a narrow junction 114, referred to as the “ostium” (FIG. 15). The precise physiological function of the LAA remains uncertain. Recent reports suggest it may maintain and regulate pressure and volume in the left atrium; modulate the hemodynamic response during states of cardiac stress; mediate thirst in hypovolemia; and/or serve as the site of release of both the peptide hormone atrial natriuretic factor (ANF), which stimulates excretion of sodium and water by the kidneys and regulates blood pressure, and stretch sensitive receptors, which regulate heart rate, diuresis, and natriuresis.

The high rate of thrombus formation in the LAA is believed to be attributable to its physical characteristics; blood easily stagnates, and thereafter clots, in the long, tubular body of the LAA or at its narrow ostium. In contrast, a right atrial appendage (RAA), which is a wide, triangular appendage connected to the right atrium by a broad ostium, is infrequently the site of thrombus formation. Thrombus formation in the LAA is further promoted by the numerous tissue folds (i.e., crenellations) on its interior surface. These crenellations are particularly hospitable to blood stagnation and clotting, especially when the heart is not functioning at maximum capacity. Thrombi formed in the LAA can re-enter the circulation upon conversion of AF to normal rhythm (i.e., cardioversion).

Certain patient subsets are considered to be at an abnormally high risk of thrombus formation. Such patients include those over seventy-five (75) years of age, as well as those presenting with a history of thromboembolism, significant heart disease, decreased LAA flow velocity, increased LAA size, spontaneous echogenic contrast, abnormal coagulation, diabetes mellitus, and/or systemic hypertension. For these high-risk patients, prophylactic intervention may be recommended.

### SUMMARY OF THE INVENTION

Some embodiments described here include a plug or insert that occludes the left atrial appendage (LAA), thus preventing blood from entering. In preferred embodiments, the plug is formed in one piece without separately movable parts, and may be monolithic. Embodiments also include a device that can maintain its position without the use of anchors that penetrate the cardiac tissues. The material used for the device is desirably highly biocompatible and may over time simply become part of the cardiac structure itself.

There are a number of aspects for devices, uses, and methods. These aspects include, without limitation, the use of a plug in a LAA; the use of a monolithic plug or other insert in a LAA; the use of a highly, bio-compatible material for the plug; the use of a porous material for the plug; the use of porous-surface silicone (PSS) for a plug; a plug for use in a LAA with a hollow portion; the use of a plug that fits into a 3 mm inner diameter catheter and yet expands to a 20 mm outer diameter, and the use of a plug with folds or grooves to aid in compression and expansion of a plug.

Clot formation during AF can also be reduced through localized delivery of agents, such as anti-platelet or anti-coagulant agents, within the LAA. Localized delivery can be accomplished by several approaches, including a coating applied to a wall, implanted one or more drug pellets, or implanting a drug delivery device. An advantage of localized drug delivery devices is that they would not obstruct or distort the LAA, as would occur with obliteration. Minimal levels of anti-coagulants and/or anti-platelet agents enter systemic circulation because the drugs are delivered for maximum benefit where and when needed. The positive effects of the drug delivery can extend to the entire left atrium, not just the LAA. The LAA is not obstructed by a device or obliterated through surgery. The risk of clot formation is reduced by delivering clot disrupting drugs locally within the LAA. The majority of proposed solutions seek to obstruct or remove the LAA significantly changing the heart structure.

Other features and advantages will become apparent from the following detailed description and drawings.

### BRIEF DESCRIPTION OF THE DRAWINGS

FIG. 1 is a perspective view of a first embodiment of a plug.

FIGS. 2 and 3 are partial perspective, partial cross-sectional views showing a plug and its insertion into a LAA.

FIGS. 4 and 5 are perspective views of other embodiments of a plug.

FIGS. 6 and 7 are perspective views showing how a hollow region can be formed in a plug, such as to produce a plug like that shown in FIG. 5.

FIGS. 8-11 are perspective views of other embodiments of a plug according to the present invention.

FIGS. 12-14 are cross-sectional and partial cross-sectional views of embodiments for applying drug delivery to the LAA.

FIG. 15 is a side view illustrating an LAA.

### DETAILED DESCRIPTION

Embodiments of the device include a single piece plug of material that is inserted into the left atrial appendage (LAA) cavity to occlude it and seal it off from the blood flow that passes through the left atrial chamber. The profile of the plug is similar to that of the LAA itself so that the device will seat in the LAA and conform to the anatomy of the LAA. Its cross section could be axisymmetric or non-uniform.

Referring to FIG. 1, a plug 10 for occluding the LAA has a flat proximal surface 12 that comes into contact with blood that flows through the left atrial chamber. The design depicted is axisymmetric and the principle cylindrical coordinate axes are labeled in the radial (R), longitudinal (X), and circumferential (θ) directions. The plug is inserted into the LAA cavity, which in the case of a completely solid plug, can completely fill the volume of the LAA cavity thereby occluding the appendage, or it can at least fill an inner portion of the LAA, such as about the innermost one-third, one-half, or two-thirds of the length of the LAA.

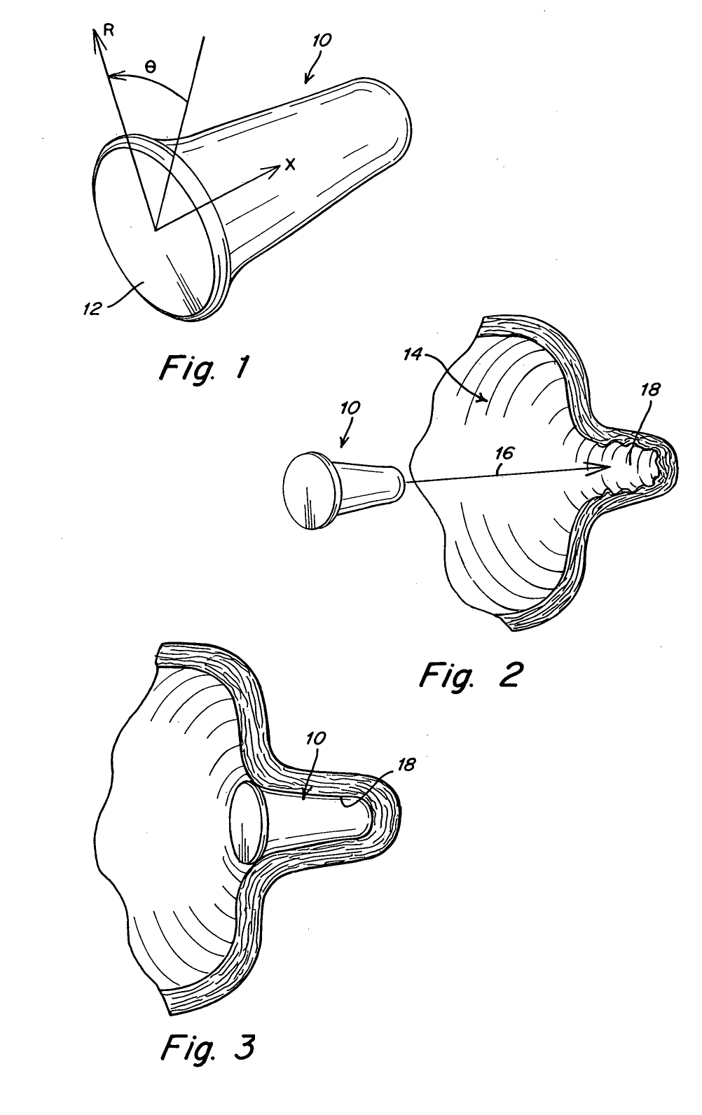

FIGS. 2 and 3 illustrate a full occlusion such that the proximal surface is at or near the ostium of the LAA 14. The larger horizontal arrow 16 illustrates how the plug is inserted into the LAA cavity 18. In FIG. 2, a left atrial chamber is shown with the LAA, which is represented by the tunnel-like cavity that emanates from the left atrial chamber. FIG. 3 shows a location of the plug following insertion into the LAA. As shown in this embodiment, the plug completely occludes the cavity of the LAA.

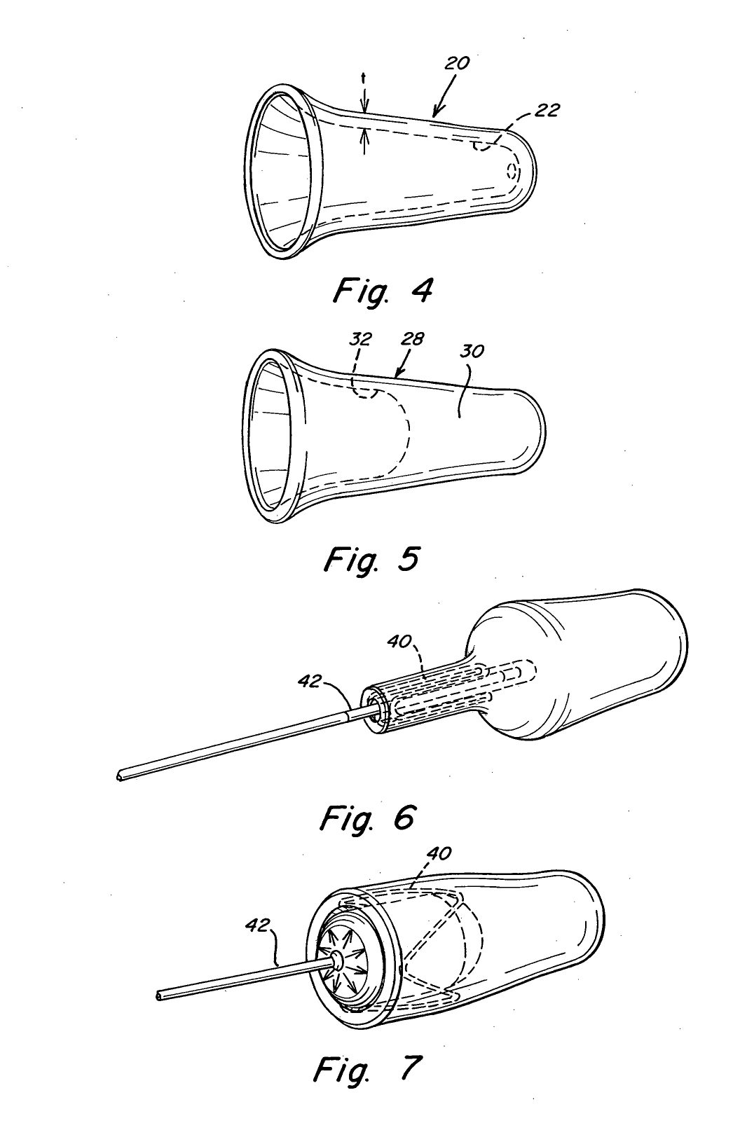

The plug can also have other configurations that range from a completely solid device as illustrated in FIG. 1 to one that is hollow and has a uniform wall thickness to a composite design that is both hollow in some parts and solid across in other parts. FIG. 4 illustrates a plug 20 that is hollow with a substantially uniform wall thickness t extending along a substantial portion of the length of the plug and defining a lumen 22, while FIG. 5 illustrates a plug 28 that has hollow and solid attributes, referred to here as a composite design. In this case, there is a uniform thickness at the proximal end extending inwardly for some distance to define a lumen 32, and then the plug is solid at portion 30. This distance where it is hollow could be, for example, about one-third, one-half, or two-thirds of the total length.

For either the hollow or composite designs, the diameter and depth of the lumen can be controlled as deemed necessary. For example, the geometry of the lumen may be designed in such a way as to minimize hemodynamic factors (e.g., flow disturbances) that may initiate thrombosis. Regardless of the geometry of the lumen, however, the profile of the plug should retain the LAA-like shape.

There are several mechanisms that can be employed, either independently or in tandem, to acutely secure the plug in LAA. These include a friction/interference fit; biologically functional adhesive; a balloon expandable annular member; the use of hooks and/or barbs; or a self-expanding annular member.

One approach to securing the plug in the LAA is to use a friction/interference fit. In this case, the dimensions of the plug are slightly oversized, e.g., 10% to 20%, relative to the LAA cavity. When the plug is inserted into the LAA cavity, the material that comprises the plug is compressed and the compressive force persists so long as the plug remains in the LAA. This residual compressive force acts in tandem with the friction that intrinsically exists at the tissue/material interface to secure the plug in place in the LAA. In this embodiment and others, the amount of friction that exists at the tissue/material interface can be controlled by modifying the surface roughness of the plug. A rougher surface generally increases the amount of friction at an interface.

The plug can be coated with an adhesive, such as a biologically functional adhesive (e.g., fibrin glue). The adhesive is applied to surfaces that will come with contact with tissue, and bonds the material of the plug to the tissue. Using biologically active adhesives can also provide additional benefits in the form of an accelerated healing response.

FIGS. 6 and 7 show another embodiment for fitting the plug. An expandable annular member (e.g. a “stent” like device 40) is incorporated into the proximal side of the plug and is dilated using a balloon catheter 42. The expandable annular member, as shown in FIG. 7, is expanded in the radial direction using a balloon dilation catheter or related means such that the outside surface of the plug makes contact with the tissue surface. This concept utilizes a plug with a lumen (which could be more like the hollow design of FIG. 4 or the composite design of FIG. 5) into which the balloon catheter can be inserted and then inflated to expand the expandable annular member.

In another embodiment, the balloon expandable annular member is replaced with a self expanding annular member that includes a shape memory material. In this case, a balloon catheter is not explicitly required to expand the proximal section of the plug.

In still another embodiment, the plug is chronically secured and relies on tissue integration into the device such that the device becomes permanently anchored in the LAA.

Another aspect of the LAA plug is the material used to construct the device. Based on the design and deployment considerations previously presented, it would be desirable for material to be biocompatible and readily accepted by the host with no adverse immunological or inflammatory responses. The material should solicit a normal and healthy healing response. The material should, over time, become integrated into the surrounding tissue milieu. Integration of the device into the tissue will ensure long term efficacy of the implant and all but eliminate the potential for embolization. The material should have an expansion ratio and/or mechanical properties that in some fashion permit the device to be advanced through a catheter lumen that is smaller than the LAA and then, when deployed, expand to plug the LAA.

One material that meets these criteria is a porous-surface silicone (PSS). PSS is a silicone-based material that has a controlled degree of porosity throughout the material. PSS material has been found to be nearly ideal matrix for tissue engineering because it is highly biocompatible and readily integratable into the tissue milieu. Animal studies have indicated that the PSS material does not induce fibrous encapsulation and neo-vascularization into the material the readily occurs. The term that is presented by the researchers to describe these phenomena is “true biointegration.”

With respect to the healing response and thrombogenicity of the plug, any and all surfaces could be modified with bioactive molecules to impart the implant with superior efficacy. Surfaces that come into contact with the circulating blood of the left atrial chamber could be coated with anti-thrombotic agents such as heparin. Tissue contacting surfaces could be coated with molecules that aid the healing response including, but not limited to, growth factors, collagen, ligands, and platelets.

The PSS is manufactured using a molding method that is amenable to fabricating components of almost any shape, size, and surface roughness. Therefore, the plug could be made in a variety of sizes and/or shapes in order to fit essentially any type of LAA.

In terms of mechanical properties, PSS, from its porous nature, is a compliant material. The compliance of PSS can also be controlled through the manufacturing process by selecting a medical grade silicone resin with the desired mechanical properties (e.g., durometer).

The plug could be delivered percutaneously via the venous circulation using common catheter practices. In an exemplary procedure, a distal end delivery catheter is delivered to the right atrium from one of several sites, such as the femoral, jugular, or brachial veins. The delivery sheath is used to deliver a need-type catheter which is used to puncture the atrial septum to gain access to the left atrium. The distal end of the delivery sheath is then passed through the atrial septum into the left atrium, and is then positioned at the LAA. The plug is collapsed into a proximal lumen of the delivery sheath and tracked to the distal end of the sheath. The plug is then deployed out of the sheath and into the LAA.

The precise aspects of the deployment of the plug are ultimately dependent upon its design. For instance, if the design of the plug utilizes the balloon expandable annular member (depicted in FIGS. 6 and 7), the deployment procedure would include expansion of the proximal portion of the plug with a balloon catheter or like accessory. Likewise, if a self-expanding annular member design were used, then an appropriate delivery system would be required.

In terms of delivery and deployment, it is desirable for the plug to be able to easily fit into a lumen of a delivery catheter, and preferably 10 French (F) or smaller delivery catheter and then, upon exiting the delivery catheter, expand to fit the LAA. The catheter could have one of a number of sizes, such as 6 F-14 F. The way the plug expands could be derived from sources already described (i.e., the intrinsic elasticity of the PSS itself or from a “stent” like device). Regardless of the source or means of expansion, the problem of how to fit the plug into the delivery catheter still remains. The size difference between the delivery catheter and the LAA can be significant; a lumen of a 10 F delivery catheter is on the order of 3 mm inner diameter whereas the LAA can be as large as 20 mm in diameter. This means that the plug should be able to fill a 20 mm diameter cavity, while also fitting into a 3 mm inner diameter lumen on delivery. With a larger diameter catheter, the plug's diameter would be reduced at least about 75% for delivery, and about 85% for delivery through a 3 mm catheter. Although PSS is highly compliant, it may not be sufficiently compliant to undergo deformations on the order of 500% or more. The plug may fit in the delivery catheter if the device is designed as an entirely hollow part as depicted in FIG. 4, but it may be much more difficult to compress a non-hollow or mostly non-hollow plug of the PSS material into the lumen of the delivery catheter.

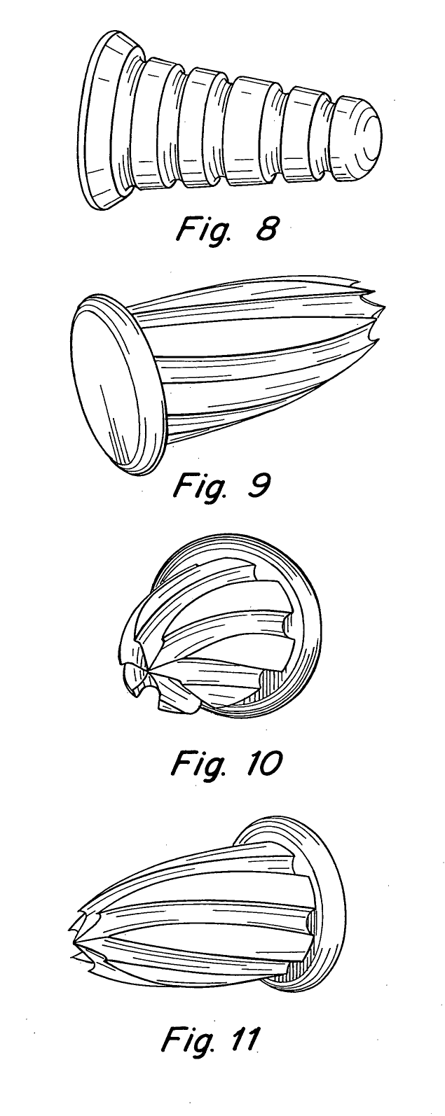

PSS can achieve elongations on the order of 400%, thereby aiding the delivery and deployment by allowing the plug to be elongated during delivery. To further aid delivery of a plug that cannot be elongated enough without further modification, the geometry and/or porosity of the device could be modified as needed to make the device easier to deliver and deploy but yet still retain the clinical utility of the device. For instance, the tissue contacting surface of the plug can have undulations as depicted in FIG. 8 that make the plug easier to compress into the lumen of the delivery catheter, or some other geometry that facilitates folding into the delivery catheter. These undulations, as shown in FIG. 8, include a series of alternating reduced diameter and full diameter sections, in this case in parallel. For instance, the plug could be fabricated with a “twister” type pattern as illustrated in FIGS. 9, 10, and 11. In these cases, the diameter is reduced further by the geometry. One or more of these shapes can have the additional benefit of improving migration resistance and reducing the risk of embolism.

The plug could be designed with any number of the previously mentioned designs to facilitate folding or collapsing of the device into the delivery sheath and subsequent deployment to the LAA.

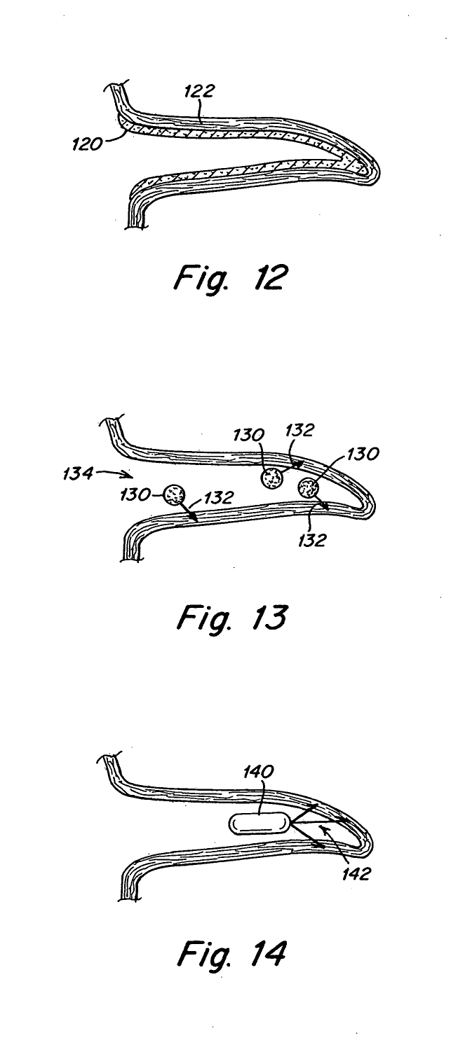

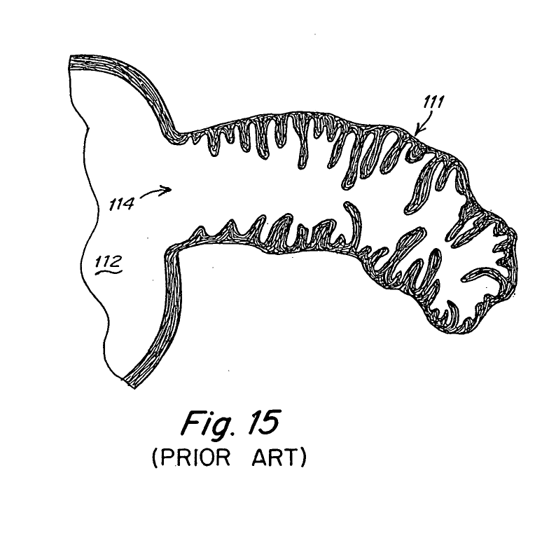

As indicated above, the plug can be coated with ant-thrombotic agents such as heparin. The potential for clot formation during AF can be reduced through localized delivery of agents, such as anti-platelet or anti-coagulant agents, within the LAA, without the use of a plug. Localized delivery can be accomplished by several approaches as described in conjunction with FIGS. 12-14.

Referring to FIG. 12, a drug release coating 120 with anti-platelet or anti-coagulant agents is applied to LAA walls 122. The coating can be delivered at the end of a device that is provided through a catheter into the left atrium, such as a physical applicator, like a small brush or sponge, or the coating can be applied to the exterior surface of a balloon that is inflated within the LAA and thereby wiped on the wall. In the case of a container, a small balloon with the coating is introduced with a plug operable by a wire; the plug is removed or withdrawn to allow the coating material to escape within the LAA. The drug coating could be applied in a liquid form or in a powder form. Rather than a catheter approach, a coating could be applied to the LAA by a surgeon, such as in the course of another procedure.

Referring to FIG. 13, in this embodiment, one or more drug release pellets 130 are implanted through anchors 132 to one or more walls of an LAA 134. These pellets can be implanted through surgery or through a catheter. Anchor 132 can be provided in the side wall of the LAA, such as through a screwing motion or some other puncture into the side wall that allows the pellets to remain in place, or the anchor can include hooks that grip the walls. These types of anchors could be made of a metal, such as nitinol or stainless steel, or a polymer. A suitable glue could also be used to mount the pellets, in addition to or instead of an anchor.

The drug released pellets can be timed to slowly release a small amount of a drug, such as an anti-coagulant, over a sustained period of time. At some point, the drug will be used up. While the anchor could be made of a non-bioresorbable material, such as nitinol, it could alternatively be made of a bioresorbable material that is slowly resorbed, so that the drug has an opportunity to be fully released before the anchor is resorbed into the tissue and/or bloodstream. Alternatively, one or more pellets with drugs could be embedded in a side wall of the LAA without anchors.

While the drug released material is described as being a pellet, it could take any shape or form that allows some form of time release, such as in the shape of a ribbon. As a further alternative, the drug could be provided as a coating on a substrate, such as a bioresorbable substrate, such that the coating is released into the system before the substrate has an opportunity to decay into the bloodstream. The substrate could be formed as a tube or tubular mesh within the walls of the LAA, like a stent. Such substrates could be delivered through a catheter or provided during surgery, such as during another procedure.

Referring to FIG. 14, in this embodiment a drug release device 140 is provided within or around the LAA, preferably held in by one or more anchors 142 similar to those described above. This drug release device can have a small valve, such as a shutter, for allowing a drug to be released. While the coating and pellets would typically have constant release mechanisms and generally not be controllable after being implanted or applied, a drug release device allows for controlled release patterns. For example, an agent can be released only when AF or abnormal cardiac patterns are detected through sensing such as that utilized in pacemakers.

The device could be triggered from another device within the body, such as an implanted pacemaker or defibrillator, or the signal could come from outside the body. Signaling can be accomplished through the use of inductive energy to a small coil in the drug delivery device, or through a radio frequency (RF) signal. An implantable pacemaker or defibrillator could be provided with a mechanism for providing a signal that is detectable by the drug delivery device. For example, small coils and other circuit components can be integrated onto very small semiconductor chips and tuned to be responsive to particular signals that could cause a valve, such as a small diaphragm or shutter, to release small amounts of agents. The agents could be provided in liquid or fine powdered form. Preferably, the release is benign if done at a time when not strictly needed.

The LAA would not be significantly distorted or damaged by the drug delivery device, and the device provides minimal obstruction.

These options can be placed within the LAA structure through minimally invasive means. The LAA is not obstructed by a device or obliterated through surgery in preferred embodiments. The risk of clotting is reduced by delivering the clot disrupting drugs locally within the LAA. Minimal levels of anti-coagulants and/or anti-platelet agents enter systemic circulation because the drugs are delivered for maximum benefit where and when needed. The positive effects of the drug would extend to the entire left atrium, not just the LAA.

Having described certain embodiments, it should be apparent that modifications can be made without departing from the scope of the invention. For example, while PSS is described as a useful material, other materials with one or more of the useful aspects that PSS has could be used, such as polyvinyl alcohol, collagen, polyurethane foam.

1. A device comprising a plug for blocking part or all of a left atrial appendage (LAA), the plug having a monolithic construction and having a proximal end and a distal end, the plug tapering from a larger diameter at the proximal end to a smaller diameter at the distal end.

2. The device of claim 1, wherein the plug has an internal cavity that extends inwardly from the proximal end for about one-third to about two-thirds of a length of the plug.

3. The device of claim 1, wherein the plug has an internal cavity that extends inwardly from the proximal end for most of a length of the plug.

4. The device of claim 1, wherein the plug has circumferential grooves formed in an outer wall.

5. The device of claim 1, wherein the plug has generally axially oriented grooves in an outer wall.

6. The device of claim 5, wherein the grooves are curved in the circumferential direction.

7. The device of claim 1, wherein the plug is made of one of porous surface silicone, polyvinyl alcohol, collagen, and polyurethane foam.

8. The device of claim 1, wherein the plug is made of porous surface silicone.

9. A method comprising providing into an LAA a plug as claimed in claim 1.

10. A method of claim 9, comprising wherein the plug has an internal cavity that extends inwardly from the proximal end, the method further comprising expanding the plug outwardly from inside the cavity.

11. The method of claim 9, further comprising securing the plug within the LAA.

12. The method of claim 11, wherein the securing includes using a biologically functional adhesive.

13. A plug comprising a substantially axisymmetric body comprising a compressible material selected from the group consisting of porous-surface silicone, polyvinyl alcohol, collagen, and polyurethane foam, wherein the plug in compressed form has a maximum diameter of less than 5 mm and wherein the plug in non-compressed form partially or wholly fills an interior volume of a left atrial appendage (LAA) of a heart.

14. The plug of claim 13, wherein the plug is made of porous-surface silicone.

15. A method comprising providing the plug of claim 13 into an LAA.

16. The method of claim 14, wherein the plug is made of porous-surface silicone.

17. A method of locally releasing one or more agents into the left atrial appendage (LAA) of a heart of a subject, the method comprising depositing a means for delivery of one or more agents on an interior wall of the LAA, and releasing the one or more agents into the interior of the LAA.

18. The method of claim 17, wherein the means for delivery includes an agent-releasing coating and the coating is applied to an interior wall of the LAA by wiping the interior wall of the LAA with an applicator that is impregnated with the drug release coating.

19. The method of claim 17, wherein the means for delivery includes one or more agent-releasing devices, each device being tethered to the interior wall of the LAA by at least one anchor implanted into the wall of the LAA.

20. The method of claim 19, wherein the rate of agent release by the one or more devices is controllable.

21. The method of claim 20, wherein the rate of agent release is controlled in a non-invasive manner by a signal originating outside the subject.

22. The method of claim 20, wherein the rate of agent release is controlled by a signal originating from a device within the subject.
